(19) United States
(12) Patent Application Publication
(10) Pub. No.: US 2011/0020519 A1
(43) Pub. Date: Jan. 27, 2011

(54) ENCAPSULATION OF OXIDATIVELY UNSTABLE COMPOUNDS

(75) Inventors: Robert G. Bowman, Woodbury, MN (US); Christopher J. Rueb, St. Paul, MN (US); John M. Finney, Eden Prairie, MN (US); William A. Hendrickson, Woodbury, MN (US); Chetan S. Rao, Austin, MN (US); Nita M. Bentley, Austin, MN (US); Richard M. Herreid, Austin, MN (US)

Correspondence Address: IPLM GROUP, P.A., POST OFFICE BOX 18455, MINNEAPOLIS, MN, 55418, US

(73) Assignee: AVEKA, Inc.

(21) Appl. No.: 12/828,577

(22) Filed: Jul. 1, 2010

Related U.S. Application Data
(63) Continuation-in-part of application No. PCT/US2009/030052, filed on Jan. 2, 2009.
Application No. PCT/US2009/030052 is a continuation-in-part of application No. PCT/US2009/030054, filed on Feb. 2, 2009.
(60) Provisional application No. 61/224,018, filed on Jul. 8, 2009.
(60) Provisional application No. 61/010,073, filed on Jan. 4, 2008.

(51) Int. Cl. A23L 3/3454; C12N 11/02; B32B 9/00; B29B 9/12

(52) U.S. Cl. 426/541; 435/177; 428/402.24; 428/402.2; 264/7

(57) ABSTRACT

An encapsulated material is formed by congealing droplets of a molten blend of oxidatively unstable material and phytosterol in a chilling gas stream to form prilled cores containing oxidatively unstable material and phytosterol, and encapsulating the prilled cores in one or more protective shell layers to form free-flowing microparticles.

### CROSS-REFERENCE TO RELATED APPLICATIONS

This application claims the benefit of the filing date of U.S. Provisional Application Ser. No. 61/224,018 filed Jul. 8, 2009, the disclosure of which is incorporated herein by reference. This application is also a continuation-in-part of copending International Application Nos. PCT/US2009/030052 and PCT/US2009/030054 each filed Jan. 2, 2009 and respectively published as WO 2009/089115 A1 and WO 2009/089117 A1, both of which claim priority from U.S. Provisional Application Ser. No. 61/010,073 filed Jan. 4, 2008, the disclosures of which are all incorporated herein by reference.

### FIELD

This invention relates to encapsulation of materials that are sensitive to oxidation.

### BACKGROUND

In the past thirty years much new information on the benefits of a healthy diet has emerged. In addition to the traditional food pyramid, vitamins and minerals, a healthy diet may include components such as soluble and insoluble fiber for promoting gastrointestinal health, phytosterols for lowering cholesterol levels and promoting heart health, antioxidants for discouraging cancer and other inflammatory diseases, and omega-3 and omega-6 polyunsaturated fatty acids (PUFAs) for promoting heart and brain health. There has been considerable commercial interest in providing deliverable forms of such components even though in many cases the component may be oxidatively unstable. PUFA-containing products or materials have introduced or announced by companies including BASF SE, Blue Pacific Flavors, GAT Food Essentials GmbH, Kerry Group PLC, Martek Biosciences Corp. and Ocean Nutrition Canada.

There is at present an ongoing and unmet need for improved methods and systems for packaging, storing or delivering PUFAs and other oxidatively unstable materials.

### SUMMARY OF THE INVENTION

The present invention provides, in one aspect, a method for protecting an oxidatively unstable material, which method comprises congealing droplets of a molten blend of oxidatively unstable material and phytosterol in a chilling gas stream to form prilled cores comprising oxidatively unstable material and phytosterol, and encapsulating the prilled cores in one or more protective shell layers to form free-flowing microparticles.

The invention provides, in another aspect, an encapsulated material comprising free-flowing microparticles containing a prilled core comprising a congealed blend of oxidatively unstable material and phytosterol, covered by at least one protective shell layer.

The disclosed encapsulated materials include at least an oxidation sensitive prilled core containing at least one oxidatively unstable material and at least one phytosterol, and at least one protective shell layer surrounding the core. The encapsulated materials may employ a multi-tiered defensive approach involving oxygen barriers, lipophilic antioxidants and hydrophilic antioxidants. The disclosed methods and materials can provide processed oils and other oxidatively unstable materials with enhanced oxidative stability and a desirable dry powder form.

### BRIEF DESCRIPTION OF THE DRAWING

FIG. 1 through FIG. 4 are schematic cross-sectional views of various encapsulated materials.

### DETAILED DESCRIPTION

Unless the context indicates otherwise the following terms shall have the following meaning and shall be applicable to the singular and plural:

The terms “a,” “an,” “the,” “at least one,” and “one or more” are used interchangeably. Thus a microcapsule that contains “a” shell may include “one or more” shells.

The term “congealed” when used with respect to a material means that the material was molten, and has been cooled or frozen without substantial solvent mass transfer or evaporation to a rigid or solid state below its melting temperature.

The term “congealing” means changing a molten material by cooling or freezing the material without substantial solvent mass transfer or evaporation, so that the material transitions from a soft or fluid state above its melting temperature to a rigid or solid state below its melting temperature.

The term “deliverable” when used with respect to an encapsulated substance means that the substance is at least partially surrounded by an additional substance that imparts one or more altered properties to the encapsulated substance, e.g., altered transport, altered flowability, altered resistance to oxidation or moisture, altered abrasion resistance, or altered performance in a commercial application (e.g., a food application).

The terms “dried” and “dryable” when used with respect to a material means that the material was or may be present in a liquid solution or dispersion, and has been or may be changed using substantial solvent mass transfer or evaporation to a rigid or solid state.

The term “drying” means changing a liquid material by substantial solvent mass transfer or evaporation to a rigid or solid state.

The terms “encapsulated material” and “microcapsule” mean particles (often but not always spherical in shape, and often but not always having a diameter of about 10 nanometers to about 5 mm which contain at least one solid core surrounded by at least one continuous membrane or shell.

The terms “fluid” and “liquid” when used in reference to a substance means that the substance has a loss modulus (G″) greater than its storage modulus (G′) and a loss tangent (tan δ) greater than 1.

The terms “gel” and “gelled” when used in reference to a substance means that the substance is deformable (viz., is not a solid), G″ is less than G′ and tan δ is less than 1.

The term “ingestible” means capable of and safe for oral administration.

The term “microsphere” means a microcapsule material whose particles contain two or more cores distributed in and surrounded by at least one continuous membrane or shell.

The term “minimally processed” when used in reference to a food means that the food meets applicable U.S. Department of Agriculture (USDA) guidelines or regulations for a minimally processed food, or in the absence of such guidelines or regulations is a food which has been subjected to a traditional process for making such food edible, to preserve it or to make it safe for human consumption, such as smoking, roasting, freezing, drying, fermenting or employing physical processes which do not fundamentally alter the raw food product or which only separate a whole, intact food into component parts, e.g. grinding meat, separating eggs into albumen and yolk, or pressing fruits to produce juices.

The term “molten” when used with respect to a blend means above the melting temperature for such blend.

The term “particulate” means a finely divided dry powder material.

The term “prilled” when used in respect to a particulate material means that the particles were formed by prilling.

The term “prilling” means forming droplets of a molten material and congealing them in a cooling gas stream to form gelled or solid microparticles.

The terms “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

The term “taste profile” means a combination of taste, flavor, consistency, odor or other sensory quality associated with eating.

The recitation of a numerical range using endpoints includes all numbers subsumed within that range (e.g., 1 to 5 includes 1, 1.5, 2, 2.75, 3, 3.80, 4, 5, etc.). The recitation of sets of upper and lower endpoints (e.g., at least 1, at least 2, at least 3, and less than 10, less than 5 and less than 4) includes all ranges that may be formed from such endpoints (e.g., 1 to 10, 1 to 5, 2 to 10, 2 to 5, etc.).

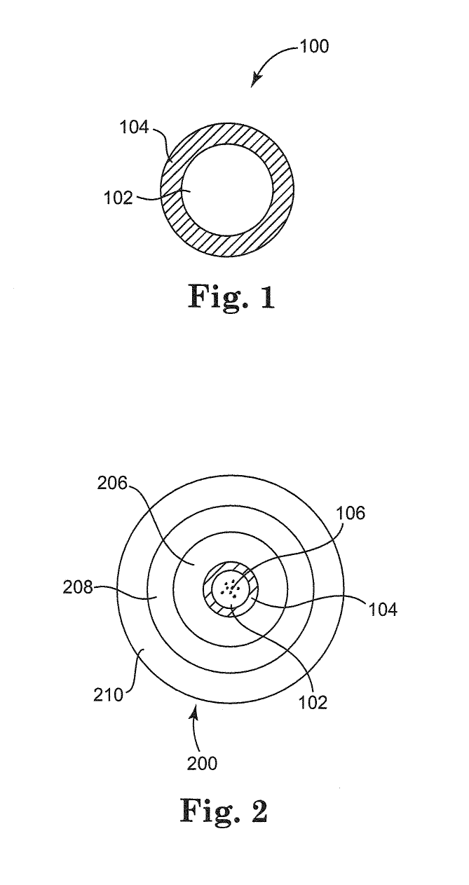

FIG. 1 shows an exemplary deliverable encapsulated material 100 including an oxidatively unstable prilled core 102 surrounded by an outer dried protective shell layer 104. Layer 104 provides a protective and water vapor transmission-resistant shell over core 102. Core 102 contains a congealed gelled or solid blend of oxidatively unstable material and phytosterol (not individually identified in FIG. 1). The oxidatively unstable material (which may, for example, be a PUFA, a refined or extracted triacylglycerol (TAG), an antioxidant, or mixture thereof) may provide a health benefit when ingested, and the phytosterol (which may, for example, be campesterol, β-sitosterol, or mixture thereof) may improve oxidation resistance of the oxidatively-sensitive material and may provide a further health benefit when ingested. The phytosterol may also contribute to one or more other properties such as structural stability (viz., keeping the core inside the shell) or steric stability (viz., increasing the shell strength). Protective shell layer 104 (which may, for example, be gelatin, agar-agar, carbohydrate, low melting wax such as stearic acid, or mixture thereof) may improve the level of oxidation resistance over that provided by the uncoated core.

FIG. 2 shows another exemplary deliverable encapsulated material 200 including oxidatively unstable core 102 surrounded by dried protective shell layer 104 as in FIG. 1. Core 102 may optionally contain dispersed solid particles 106 which may alter the properties of core 102 or layer 104, or may provide other features to encapsulated material 100. Particles 106 may be formed for example from solids including calcium salts, alginic acid and salts thereof including sodium or calcium alginate, chelating agents including citric acid, or antioxidants including ascorbic acid. Shell 104 is surrounded by an intermediate hydrocolloid shell 206 made for example from alginate, an intermediate fiber/carbohydrate shell 208 made for example from a mixture of maltodextrin, sucrose, trehalose and starch, and an outer protective layer 210 made for example from a mixture of lipid, fiber and protein. Layers or shells 104, 206 and 208 may optionally contain at least one phytosterol. The various layers shown in FIG. 2 are merely exemplary and may be rearranged, combined into fewer layers, augmented with additional layers or made from other ingredients or mixtures of ingredients. Doing so may facilitate formation of encapsulated materials which maintain, preserve or protect the prilled core and keep oxygen and if desired one or both of water or light away from the core.

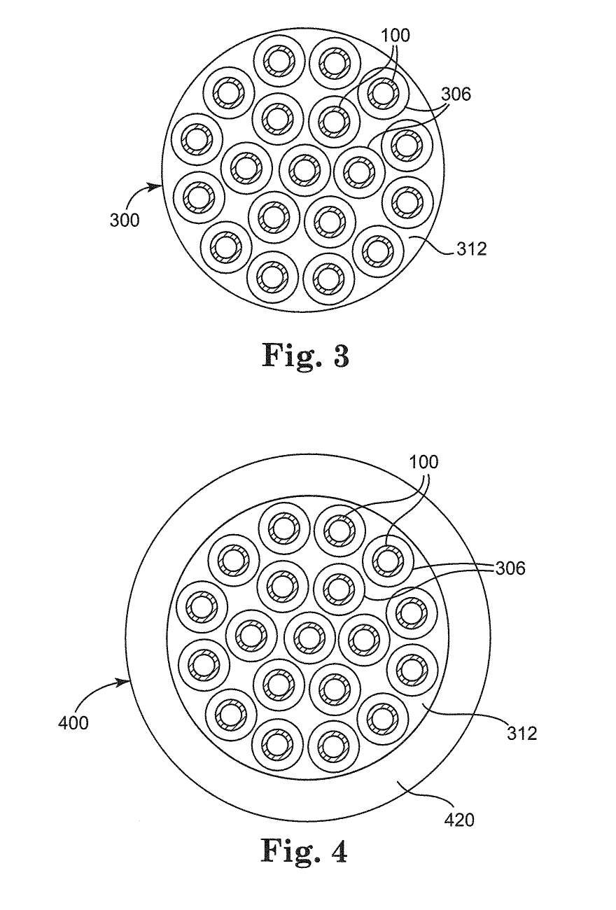

FIG. 3 shows another exemplary deliverable encapsulated material in the form of a microsphere 300 including a plurality of oxidatively unstable core particles 100 similar to those shown in FIG. 1 surrounded by intermediate hydrocolloid shells 306 made for example from gelatin or alginate. The particles 100 and their shells 306 may be dispersed in a protective matrix 312 made for example from a mixture of maltodextrin, sucrose, starch, ascorbic acid and oat fiber. The shells 306 or matrix 312 may optionally contain at least one phytosterol.

FIG. 4 shows another exemplary deliverable encapsulated material in the form of a microsphere 400 including a plurality of oxidatively unstable core particles 100 and surrounding intermediate hydrocolloid shells 306 dispersed in a protective matrix 312, and surrounded by a protective wax-containing shell 420. Shell 420 may include a variety of other ingredients, e.g., soluble fibers, lipid soluble materials including tocopherols, and dispersed water-soluble particulates including ascorbic acid and citric acid. Shell 420 may optionally contain at least one phytosterol.

A variety of oxidatively unstable materials may be used in the core. Exemplary oxidatively unstable materials include acidulants, animal products, antioxidants, carotenoids, catalysts, drugs, dyes, enzymes, flavors, fragrances, lutein, lycopene, metal complexes, natural colors, nutraceuticals, pigments, polyphenolics, processed plant materials, metabiotics, probiotics, proteins, PUFAs, squalenes, sterols other than phytosterols, tocopherol, tocotrienol, TAGs, vitamins (e.g., fat-soluble vitamins)), unsaturated organic compounds (e.g., unsaturated rubbers and unsaturated oils) and mixtures thereof. PUFAs, antioxidants, sterols other than phytosterols and TAGS are of particular interest. The oxidatively unstable material may for example represent about 2 to about 96 wt. %, about 5 to about 96 wt. %, about 20 to about 96 wt. % or about 40 to about 96 wt. % of the encapsulated material.

For oxidatively unstable materials that normally are liquids at the desired use temperature (e.g., at room temperature or about 25° C.), it may be desirable to gel the liquid in order to facilitate prilling. For example, core materials based on liquids may be gelled as described in U.S. Pat. No. 6,858,666 B2 (Hamer et al.) where an oxidatively unstable liquid oil is heated in the presence of a suitable gelation agent to melt and dissolve the gelation agent in the continuous oil phase. The resultant solution may then be atomized and congealed to form prilled cores. The amount of gelation agent(s) may for example range from about 1 to about 90 wt. % of the core weight. Additional exemplary gelled core particles based on PUFAs may be formed by combining a PUFA with a phytosterol to form triglyceride-recrystallized phytosterols as in U.S. Pat. Nos. 6,638,547 B2 (Perlman et al.) and 7,144,595 B2 (Perlman et al.) and U.S. Patent Application Publication No. 2006/0251790 A1 (Perlman et al.). Some antioxidants, e.g., Vitamin E, may also help convert a liquid core material to a gel. Oxidatively unstable materials that normally are liquids at the desired use temperature may also be made by carrying out prilling at a sufficiently low temperature to enable formation of gelled or solid prilled cores, followed by encapsulation at a sufficiently low temperature to enable formation of the protective shell layer.

Exemplary PUFAs include those found in fish and various grain products, e.g., fish oil, halibut, herring, mackerel, menhaden, salmon, algae, chia, flaxseed and soybeans. PUFAs based on deodorized fish oils containing substantial amounts of omega-3 or omega-6 fatty acids are of particular interest. Exemplary omega-3 fatty acids include all-cis-7,10,13-hexadecatrienoic acid (16:3ω3), α-linolenic acid (ALA, 18:3ω3), stearidonic acid (STD, 18:4ω3), eicosatrienoic acid (ETE, 20:3ω3), eicosatetraenoic acid (ETA, 20:4ω3), eicosapentaenoic acid (EPA, 20:5ω3), docosapentaenoic acid (DPA, 22:5ω3), docosahexaenoic acid (DHA, 22:6ω3), tetracosapentaenoic acid (24:5ω3), tetracosahexaenoic acid (nisinic acid, 24:6ω3) and mixtures thereof. Exemplary omega-6 fatty acids include linoleic acid (18:2ω6), gamma-linolenic acid (18:3ω6), eicosadienoic acid (20:2ω6), dihomo-gamma-linolenic acid (20:3ω6), arachidonic acid (20:4ω6), docosadienoic acid (22:2ω6), adrenic acid (22:4ω6), docosapentaenoic acid (22:5ω6), calendic acid (18:3ω6) and mixtures thereof.

Exemplary antioxidants include menaquinone (vitamin K₂), plastoquinone, retinol (vitamin A), vitamin D, vitamin E, phylloquinone (vitamin K₁), tocopherols, tocotrienols (e.g., α, β, γ and δ-tocotrienols), ubiquinol, and ubiquione (Coenzyme Q10)); and cyclic or polycyclic compounds including acetophenones, anthroquinones, benzoquiones, biflavonoids, catechol melanins, chromones, condensed tannins, coumarins, flavonoids, hydrolyzable tannins, hydroxycinnamic acids, hydroxybenzyl compounds, isoflavonoids, lignans, naphthoquinones, neolignans, phenolic acids, phenols (including bisphenols and other sterically hindered phenols, aminophenols and thiobisphenols), phenylacetic acids, phenylpropenes, stilbenes and xanthones. Additional cyclic or polycyclic antioxidant compounds include apigenin, auresin, aureusidin, Biochanin A, capsaicin, catechin, coniferyl alcohol, coniferyl aldehyde, cyanidin, daidzein, daphnetin, delphinidin, emodin, epicatechin, eriodicytol, esculetin, ferulic acid, formononetin, gernistein, gingerol, 3-hydroxybenzoic acid, 4-hydroxybenzoic acid, 3-hydroxycoumarin, juglone, kaemferol, lunularic acid, luteolin, malvidin, mangiferin, 4-methylumbelliferone, mycertin, naringenin, pelargonidin, peonidin, petunidin, phloretin, p-hydroxyacetophenone, (+)-pinoresinol, procyanidin B-2, quercetin, resorcinol, rosmaric acid, salicylic acid, scopolein, sinapic acid, sinapoyl-(S)-maleate, sinapyl aldehyde, syrginyl alcohol, telligrandin II, umbelliferone and vanillin. Antioxidants may also be obtained from plant extracts, e.g., from blackberries, blueberries, black carrots, chokecherries, cranberries, black currants, elderberries, red grapes and their juice, hibiscus, oregano, purple sweet potato, red wine, rosemary, strawberries, tea (e.g., black, green or white tea), and from various plant ingredients as ellagic acid. Additional exemplary antioxidants include carotenoids including hydrocarbons such as hexahydrolycopene, lycopersene, phtyofluene, torulene and α-zeacarotene; alcohols such as alloxanthin, cynthiaxanthin, cryptomonaxanthin, crustaxanthin, gazaniaxanthin, loroxanthin, lycoxanthin, pectenoxanthin, rhodopin, rhodopinol and saproxanthin; glycosides such as oscillaxanthin and phleixanthophyll; ethers such as rhodovibrin and spheroidene; epoxides such as citroxanthin, diadinoxanthin, foliachrome, luteoxanthin, mutatoxanthin, neochrome, trollichrome, vaucheriaxanthin and zeaxanthin; aldehydes such as rhodopinal, torularhodinaldehyde and wamingone; ketones such as canthaxanthin, capsanthin, capsorubin, cryptocapsin, flexixanthin, hydroxyspheriodenone, okenone, pectenolone, phoeniconone, phoenicopterone, phoenicoxanthin, rubixanthone and siphonaxanthin; esters such as astacein, fucoxanthin, isofucoxanthin, physalien, siphonein and zeaxanthin dipalmitate; apo carotenoids such as β-apo-2′-cartoenal, apo-2-lycopenal, apo-6′-lycopenal, azafrinaldehyde, bixin, citranaxanthin, crocetin, crocetinsemialdehyde, crocin, hopkinsiaxanthin, methyl apo-6′-lycopenoate, paracentrone and sintaxanthin; nor and seco carotenoids such as actinioerythrin, β-carotene, peridinin, pyrrhoxanthininol, semi-α-carotenone, semi-β-carotenone and triphasiaxanthin; retro and retro apo carotenoids such as eschscholtzxanthin, eschscholtzxanthone, rhodoxanthin and tangeraxanthin; higher carotenoids such as decaprenoxanthin and nonaprenoxanthin; secondary aromatic amines; alkyl and arylthioethers; phosphates and phosphonites; zinc-thiocarbamates; benzofuranone lactone-based antioxidants; nickel quenchers; metal deactivators or complexing agents; and the like. Commercially available antioxidants include butylated hydroxyanisole (BHA), 2,6-di-t-butyl cresol (BHT), 2,2′-methylene bis(6-t-butyl-4-methyl phenol) (available as VULKANOX™ BKF from Bayer Inc., Canada), 2,2′-thio bis(6-t-butyl-4-methyl phenol), tert-butyl hydroquinone, di-tert-butyl hydroquinone, di-tert-amyl hydroquinone, methyl hydroquinone, p-methoxy phenol, tetrakis[methylene-3-(3′,5′-di-tert-butyl-4′-hydroxyphenyl)propionate]methane, N-(2-aminoethyl)-3-[3,5-bis(tert-butyl)-4-hydroxyphenyl]propanamide, 5,7-di-tert-butyl-3-(3,4,-dimethylphenyl)-3H-benzofuran-2-one, dilauryl thiodipropionate, dimyristyl thiodipropionate, tris(nonylphenyl) phosphite, and the like, and mixtures thereof. The antioxidants 2,2′-methylene bis(6-t-butyl-4-methyl phenol) and N-(2-aminoethyl)-3-[3,5-bis(tert-butyl)-4-hydroxyphenyl]propanamide may be preferred for some applications, with the latter antioxidant being especially desirable because it includes a reactive amino group which may enable covalent incorporation into a suitably reactive core or shell. Antioxidants may, for example, suppress, reduce, intercept, or eliminate destructive radicals or chemical species that promote the formation of destructive radicals which would otherwise lead to more rapid oxidative degradation of the encapsulated material or components thereof.

Exemplary sterols other than phytosterols include cholesterol, steroidal hormones such as testosterone, vitamins such as D vitamins, eicosanoids (e.g., hydroxyeicostetraones, prostacyclins, prostaglandins and thromboxanes, leukotrienes, lipoxins, resolvins, isoprostanes and jasmonates.

Exemplary TAGs include those found in algae oil, almond oil, beef tallow, butterfat, canola oil, chia oil, cocoa butter, coconut oil, cod liver oil, corn oil, cottonseed oil, flaxseed oil, grape seed oil, lard, olive oil, palm oil, peanut oil, safflower oil, sesame oil, soybean oil, sunflower oil, and walnut oil.

A variety of phytosterols may be used in the core. Exemplary phytosterols include campesterol, stigasterol, β-sitosterol, Δ5-avenosterol, Δ7-stigasterol, Δ7-avenosterol, brassicasterol or mixtures thereof. Non-esterified phytosterols are preferred for use in the disclosed method, although esterified phytosterols may also be employed. The phytosterol may for example represent about 0.5 to about 96 wt. %, about 3.5 to about 96 wt. %, about 5 to about 80 wt. % or about 5 to about 60 wt. % of the encapsulated material. Cores containing a combination of phytosterol and omega-3 fatty acids are especially preferred. Exemplary such combinations include the formulations shown below in Table 1:

TABLE 1

| Phytosterol, mg | Omega-3, mg | Phytosterol/Omega-3 |
|---|---|---|
| 650 | 32 | 20.3 (325:16) |
| 650 | 50 | 13.0 |
| 400 | 50 | 8.0 |
| 650 | 100 | 6.5 |
| 650 | 150 | 4.3 |
| 400 | 100 | 4.0 |
| 400 | 150 | 2.7 (8:3) |

For example, the Table 1 formulations may be added to food products or administered as is (e.g., in one or more capsules) to provide recommended daily servings meeting U.S. Food and Drug Administration (FDA) guidelines.

The core may comprise, consist of or consist essentially of oxidatively unstable material and phytosterol. The core may include additional ingredients having limited or no susceptibility to oxidation, e.g., caveolins, phospholipids, micelle stabilizers, soluble or insoluble fibers, various oils, various salts, and mixtures thereof. Phospholipids and micelle stabilizers may be of particular interest. Exemplary phospholipids include natural or chemically modified phospholipids, e.g., alkylphosphocholines (viz., synthesized phospholipid-like molecules), cardiolipin, dipalmitoylphosphatidylcholine, glycerophospholipid, lecithin, phosphatidic acid, phosphatidylcholine, phosphatidylethanolamine, phosphatidylglycerol, phosphatidylinositol, phosphatidylinositol 3-phosphate, phosphatidylinositol (3,4)-bisphosphate, phosphatidylinositol (3,5)-biphosphate, phosphatidylinositol (3,4,5)-triphosphate, phosphatidylmyo-inositol mannosides, phosphatidylserine, sphingomyelin, sphingosyl phosphatide and mixtures thereof. An exemplary commercially available phospholipid is ULTRALEC F™ deoiled lecithin from Archer Daniels Midland Co. (Decatur, Ill.). Exemplary micelle stabilizers (some of which are phospholipids) include cardolipin, digalactosyldiacylglycerols, monogalactosyldiacylglycerols, phosphatidylcholine, phosphatidylethanolamine, phosphatidylserine, phosphatidylinositol and sphingolipids and mixtures thereof.

The core may be hollow or solid and desirably is solid. The core may be spherical or other than spherical (e.g., oblong) and desirably is spherical. The core desirably has a low melt viscosity, low specific heat capacity and low heat of melting. The core may for example have a melting temperature below about 50° C., below about 100° C., below about 150° C., below about 200° C. or below about 250° C. Exemplary core microparticles may for example have particle diameters from about 10 nanometers to about 5,000 micrometers, about 1 micrometer to about 1,000 micrometers, or about 10 micrometers to about 500 micrometers. The core may for example represent at least about 5 wt. %, at least about 20 wt. % or at least about 30 wt. % of the encapsulated material. Desirably the core is greater than 30 wt. % of the encapsulated material, e.g., at least about 40 wt. % or at least about 50 wt. %.

The core may be formed using a variety of types of prilling equipment. Representative such equipment includes systems from Buchi Corporation, GEA Niro and Armfield Ltd. Industrial Food Technology. Prilling may also be performed by modifying equipment used for other purposes. Appropriate operating parameters for prilling ordinarily will be established empirically, and may vary depending on factors such as the desired core material, desired throughput, ambient conditions and chosen prilling equipment.

A variety of microencapsulating materials may be used in the disclosed encapsulated materials to form protective shell layer(s), sometimes also referred to as coatings or membranes, surrounding the core(s), or as additives in a protective shell layer. Exemplary such materials provide a barrier to one or more of oxygen, water, light or other oxidation promoters, and include dryable materials and prillable materials. The microencapsulating materials may comprise, consist of or consist essentially of natural, semisynthetic (viz., chemically modified natural materials) or synthetic materials. Exemplary natural materials include gum arabic, agar agar, agarose, maltodextrins, alginic acid and salts thereof including sodium or calcium alginate, fats and fatty acids, cetyl alcohol, collagen, chitosan, lecithins, gelatin, albumin, shellac, polysaccharides including starch or dextran, polypeptides, protein hydrolyzates, sucrose and waxes. Oleosins as described in copending PCT Application No. PCT/US2009/030052 or phospholipids as described in copending PCT Application No. PCT/US09/30054 may be employed in the protective shell layer. Exemplary semisynthetic materials include chemically modified celluloses including cellulose esters and ethers (for example cellulose acetate, ethyl cellulose, hydroxypropyl cellulose, hydroxypropyl methyl cellulose and carboxymethyl cellulose) and chemically modified starches including starch ethers and esters (for example, CAPSUL™ modified starch from National Starch). Exemplary synthetic materials include polymers (for example, polyacrylates, polyamides, polyvinyl alcohol, polyvinyl pyrrolidone, polyureas and polyurethanes). Exemplary commercial microcapsule products (the shell materials for which are shown in parentheses) include Hallcrest Microcapsules (gelatin, gum arabic), Coletica THALASPHERES™ (maritime collagen), Lipotec MILLICAPSELN™ (alginic acid, agar agar), Induchem UNISPHERES™ (lactose, microcrystalline cellulose, hydroxypropylmethyl cellulose), Unicerin C30 (lactose, microcrystalline cellulose, hydroxypropylmethyl cellulose), Kobo GLYCOSPHERES™ (modified starch, fatty acid esters), SOFTSPHERES™ (modified agar agar) and Kuhs Probiol NANOSPHERES™. Preferred dryable microencapsulating materials include gelatin, agar-agar, alginates, pectin, starch, carbohydrates and mixtures thereof. Preferred prillable microencapsulating materials include low melting waxes such as triglyceride waxes and mixtures thereof, e.g., bees wax, canola wax, Carnauba wax, Candelilla wax, castor wax, stearic acid, and commercially available triglyceride waxes including ASTOR™ and A-C™ waxes from Honeywell International Inc., BE SQUARE™ waxes from Baker Petrolite, DRITEX™-C and DRITEX-S waxes from ACH Food Companies, Inc. and DYNASAN™ waxes from Dynamit Nobel, Inc. The protective shell layer may for example represent about 2 to about 90 wt. %, about 3.5 to about 90 wt. %, about 10 to about 80 wt. % or about 30 to about 60 wt. % of the encapsulated material.

The protective shell layer may be in direct contact with a surface of the core, or may be in direct contact with an intermediate layer located between a surface of the core and the protective shell layer. The latter configuration may however have a reduced core content or core loading for a given particle size. The protective shell layer may be covered by one or more additional layers. If the encapsulated material is not required to be ingestible, then the outer and if desired inner layers may be ingestible or not as desired, whereas for ingestible encapsulated materials at least the outermost layer is ingestible. Exemplary outer layers include a water-dispersible oxygen-barrier layer, hydrocolloid layer, lipophilic layer or any combination thereof. For example, a hydrocolloid or HC layer made using a natural or chemically-modified hydrocolloid material, e.g., an alginate, may facilitate upper gastrointestinal (UGI) tract bypass when the disclosed encapsulated materials are orally administered to mammalian subjects. Further details regarding exemplary HC layers may be found in the above-mentioned PCT Application Nos. PCT/US2009/030052 and PCT/US09/30054. A particularly useful layer, especially over the protective shell layer, is a fiber/carbohydrate/protein or FPC layer made using a fiber-, carbohydrate or protein-containing film-forming material. Exemplary FPC layers may be formed from at least one of dietary fiber (e.g., food grade fiber), a simple carbohydrate (e.g., a monosaccharide or disaccharide such as a sugar), or a protein. Further details regarding exemplary FPC layers may be found in the above-mentioned PCT Application Nos. PCT/US2009/030052 and PCT/US09/30054.

The various layer ingredients discussed above may arrange themselves into separate layers around the cores (for example due to reasons such as stereochemistry, surface energy, oleophilicity, oleophobicity, hydrophilicity or hydrophobicity). The layer ingredients may in some embodiments form a matrix of ingredients in a single shell layer surrounding the cores.

In some embodiments the protective shell layer or other layers may contain one or more antioxidants. Exemplary antioxidants include those discussed above in connection with the core. Some antioxidants may be used as core stabilizers and as shell stabilizers. The disclosed encapsulated materials may contain a variety of other adjuvants, including chelating agents, surfactants, UV absorbers and other ingredients or additives that will be familiar to persons having ordinary skill in the microencapsulation art. Further details regarding exemplary adjuvants may be found in the above-mentioned PCT Application Nos. PCT/US2009/030052 and PCT/US09/30054. The disclosed encapsulated materials may also include absorbents, dehydrators, flow aids and other agents that may assist in pouring, storing or dispensing the encapsulated materials or in mixing them with other materials. The agent may in some embodiments form a coating over an outer layer, in effect representing an additional shell, and may in other embodiments be an additive included in an outer layer. The agent may change the surface energy of the encapsulated material, absorb excess oil, or serve other functions. Further details regarding exemplary agents may be found in the above-mentioned PCT Application Nos. PCT/US2009/030052 and PCT/US09/30054. The adjuvant or agent may for example represent about 0.1 to about 5 wt. % of the encapsulated material.

A variety of exemplary structures and methods may be used to form the disclosed encapsulated materials. If made using a dryable microencapsulating material, the protective shell layer may for example contain less than 8%, less than 6%, less than 5%, less than 4%, less than 3%, less than 2% or less than 1% of available water. The desired dryness level may be reached by removing water (e.g., if the protective shell layer is formed using an aqueous carrier or solvent) or by adding water (e.g., if the protective shell layer is formed using an organic carrier or solvent) after or during formation of the disclosed encapsulated material. In one exemplary embodiment the prilled cores are formed and then dispersed into an emulsion whose continuous phase contains ingredients capable of forming one and optionally several layers surrounding the core microparticles. The emulsion may be processed (e.g., spray dried) to convert the emulsion into microcapsules having at least one dried protective layer surrounding the cores. In another exemplary embodiment the prilled cores are formed and then coated with gelatin using any of a variety of methods including spray drying, incipient wetness, or coating in a WURSTLR™ air suspension coater (from Glatt GmbH). For example, gelatin could be added to water heated at or near boiling to dissolve the gelatin, then cooled to near ambient temperature. The prilled cores could be suspended in the gelatin/water mixture, and the mixture could be spray dried to form the disclosed free-flowing microparticles.

If made using a prillable microencapsulating material, the microencapsulating material may for example be in fluid form at an elevated temperature (e.g., at above 30° C.) and in solid form when cooled to a lower temperature. The microencapsulating material desirably has a lower melting point (e.g., a melting point at least 2° C., at least 5° C., at least 10° C., or at least 20° C. lower) than that of the prilled core. This will facilitate dispersing the cores into a molten sample of the microencapsulating material and reprilling to provide the disclosed microparticles. In one embodiment the prilled cores are suspended in a low melting wax such as stearic acid, and then re-prilled to form the disclosed free-flowing microparticles. In another exemplary embodiment the prilled cores are dry-blended with a micronized low melting wax such as micronized stearic acid and brief heat or high shear forces are applied to melt the wax onto the cores.

The various applied layers may be reacted with a variety of materials to alter some or all of the layer characteristics. This may be carried out using a variety of reaction schemes, materials and other measures. For example, a Maillard reaction between proteins and reducing sugars may be used to alter a layer containing protein or a layer containing a reducing sugar by exposing such layers to reducing sugar or protein, respectively, in the presence of sufficient heat to promote a browning reaction. Hydrocolloid (e.g., alginate layers) may be crosslinked, e.g., by inclusion of a suitable calcium salt source in the hydrocolloid layer, in an adjacent layer or in the core.

An exemplary encapsulated material may for example be made using an oxidatively unstable material (e.g., a TAG or PUFA) to which has been added a phytosterol and optionally an antioxidant (e.g., tocopherol, lycopene or tocotrienols), chelating agents, or dispersed calcium carbonate or calcium sulfate. The core may be formed by heating the core ingredients to an appropriate temperature above their melting point, for example to about 70-80° C., then atomizing the mixture in a grilling apparatus and rapidly congealing the resulting droplets in a chilled gas stream (e.g., chilled air or liquid nitrogen) to form prilled beads. The beads may be coated with a protective shell layer which may be made from a variety of materials and formed using a variety of techniques. The formation of a protective shell layer may be repeated several (e.g., one to four) times. HC shell (HCS) layers may be formed, for example from an aqueous sodium alginate hydrocolloid solution to which a variety of other materials may also be added. FCPS layers may be formed, for example by adding fibers such as insoluble fiber or carboxymethyl cellulose (CMC) fibers and optional additives to a solution containing water-soluble antioxidants and reducible sugars. The resulting mixture may be formed into encapsulated materials, e.g., by adding the prilled cores to the solution and spray drying to form FCP-coated microparticles. In a preferred process the resulting spray dried product is added to a melt for prilling or otherwise converted in order to form an outer lipophilic shell or LPS over an FCP-coated core. Separation of microcapsules by centrifugation or filtration and drying to a dry state may also or instead be used to form various layers.

Using these various general processes for manufacture, a variety of different materials, layers and constructions can be used to provide a variety of encapsulated materials. Set out below in Table 2 are several non-limiting exemplary structural components, ingredients and functions for use in such processes. The terms “AI” and “AO” in Table 2 respectively refer to an “active ingredient” and an “antioxidant”, functions which in some cases may be performed by the same material. Typically an AI or AO will be carried and protected by the core, protective shell, HCS, FCPS or other layer until such time as the AI or AO may be delivered to an intended host or site for a subsequent designed use. Other abbreviations are identified in the footnotes to Table 2. To simplify the table appearance, the first row for each new structural component (e.g., Core, protective shell, etc.) includes the structural component label, and subsequent rows showing other materials for use in or as such structural component do not explicitly show the structural component label but are deemed to have been so labeled.

TABLE 2

| Structural | | |
|---|---|---|
| Component | Ingredient | Function |
| Core | PUFA¹ | AI² |
| | Vegetable Oil | AI or AO³ |
| | Lycopene | AI or AO |
| | Lutein | AI or AO |
| | Tocopherol | AI or AO |
| | Phytosterol | AI, organogellation agent or AO |
| | BHT⁴ | AI or AO |
| | Calcium Compound | Crosslinking agent for HCS⁵ |
| | Citric Acid | Metal chelating agent for |
| | | prooxidants or AI |
| | EDTA⁶ Salt | Metal chelating agent for |
| | | prooxidants |
| | Phytosterol | AI, oxygen Barrier or AO |
| Phospholipid | Phospholipid | Liposome shell, core stabilizer |
| Shell | | and AO |
| | Phytosterol | Liposome shell stabilizer or AO |
| | Oleosin | Liposome shell stabilizer |
| Hydrocolloid | Alginate | Shell Matrix, UGI⁷ bypass and |
| Shell | | oxygen barrier |
| | CMC⁸ | Shell, oxygen barrier |
| | Insoluble Fiber | Shell, oxygen barrier |
| | HPMC⁹ | Shell, oxygen barrier |
| | Anthocyanin | AO |
| | BHT | AO |
| | Lutein | AO |
| | Lycopene | AO |
| | Tocopherol | AO |
| | Carbohydrate | AI |
| | Dextrose | Reducible sugar for Maillard |
| | | reaction and carbohydrate |
| | Fructose | Reducible sugar for Maillard |
| | | reaction and carbohydrate |
| | Lactose | Reducible sugar for Maillard |
| | | reaction and carbohydrate |
| | Sucrose | Nonreducible sugar and |
| | | carbohydrate |
| | Trehalose | Nonreducible sugar and |
| | | carbohydrate |
| | Casein | Protein for Maillard reaction |
| | WPC¹⁰ | Protein for Maillard reaction |
| | Phytosterol | AI, oxygen Barrier or AO |
| Fiber/ | Pectin | Soluble fiber for UGI bypass |
| Carbohydrate/ | | |
| Protein Shell | | |
| | Insoluble Fiber | Oxygen barrier |
| | Alginate | Matrix, soluble fiber, oxygen |
| | | barrier |
| | Starch | Matrix, soluble fiber, oxygen |
| | | barrier |
| | Dextrose | Reducible sugar for Maillard |
| | | reaction and carbohydrate |
| | Fructose | Reducible sugar for Maillard |
| | | reaction and carbohydrate |
| | Lactose | Reducible sugar for Maillard |
| | | reaction and carbohydrate |
| | Sucrose | Nonreducible sugar and |
| | | carbohydrate |
| | Trehalose | Nonreducible sugar and |
| | | carbohydrate |
| | Casein | Protein for Maillard reaction |
| | Gelatin | Matrix protein for Maillard |
| | | reaction, oxygen barrier |
| | WPC | Protein for Maillard reaction |
| | Whey | Reducible sugar and protein |
| | | for Maillard reaction |
| | Lycopene | AO |
| | Lutein | AO |
| | Tocopherol | AO |
| | BHT | AO |
| | Phytosterol | AI, oxygen barrier or AO |
| Lipophilic | Hydrogenated Oil | Oxygen barrier, AO |
| Shell | | |
| | Phytosterol | AI, oxygen barrier or AO |

¹PUFA is polyunsaturated fatty acid.
²AI is active ingredient.
³AO is antioxidant.
⁴BHT is 2,6-di-t-butyl cresol.
⁵HCS is hydrocolloid shell.
⁶EDTA is ethylenediaminetetraacetic acid.
⁷UGI is upper gastrointestinal tract.
⁸CMC is carboxymethylcellulose.
⁹HPMC is hydroxypropylmethylcellulose.
¹⁰WPC is whey protein concentrate.

For encapsulated materials having a core surrounded by a single protective shell layer, the core:shell weight ratio may for example range from about 20:1 to about 1:20, about 10:1 to about 1:10, about 8:1 to about 1:1, or about 2:1 to about 2:3. For encapsulated materials having a core surrounded by four shell layers (e.g., a core having protective shell, HCS, FCPS and LPS layers), the core may for example represent about 5 to about 70, about 5 to about 60 or about 10 to about 40 wt. % of the total encapsulated material weight. Set out below in Table 3 are exemplary constructions showing core and layer amounts (expressed in parts by weight) for a variety of encapsulated materials containing protective shell-coated cores, and additional layers each of which may also serve as a protective layer, together with the approximate core weight percent.

TABLE 3

| Layer\Example | A | B | C | D | E | F |
|---|---|---|---|---|---|---|
| Core | 80 | 80 | 80 | 80 | 80 | 80 |
| Protective Shell | 20 | 20 | 20 | 20 | 15 | 20 |
| Alginate Shell | 20 | 20 | 20 | 20 | 20 | 20 |
| Fiber/Carbohydrate/ | 120 | 80 | 40 | 120 | 20 | 120 |
| Protein Shell | | | | | | |
| Lipophilic Shell | 240 | 240 | 240 | 0 | 0 | 1400 |
| Percent Core | 16% | 18% | 20% | 33% | 60% | 5% |

The data in Table 3 show encapsulated materials with four shell layers containing about 5-60 wt. % core content. By varying the presence or absence of the various layers and their ingredients and relative amounts, encapsulated materials having a variety of properties can be formed. For example, if the lipophilic shell is eliminated and a fiber/carbohydrate/protein shell containing mainly a soluble fiber such as pectin or alginate is employed, a taste-masked encapsulated material with UGI bypass characteristics may be prepared. If a phytosterol-containing lipophilic shell is employed, a high temperature encapsulated material with an AO shell may be prepared for use in baked products and baking applications. Encapsulated materials whose cores or lipophilic shells contain organogels, and encapsulated materials with lipophilic shells containing hydrogenated oils crystallized in the beta form, may provide oxygen barrier or zero order (viz., concentration-independent) release characteristics.

Oxidative stability may be evaluated using a variety of tests. Simple but sensitive subjective tests such as olfactory tests or taste tests will suffice for many applications. For example, a subjective taste profile measurement may be performed using a panel of at least five people and the following six point scale:
0=no difference
1=very slight difference
2=slight difference
3=moderate difference
4=large difference
5=extreme difference

The panel members may be asked to sample unaged or aged (e.g., for one, three, six or twelve months) food products containing the disclosed encapsulated materials, and to compare their tastes using the six point scale. An aged food product having a 0 or 1 rating may be regarded as having a minimal off taste profile.

A variety of objective tests may also be employed, including accelerated oxidative stress tests such as solid phase micro extraction (SPME) at an elevated temperature, e.g., 50° C. in an oxidizing atmosphere such as pure oxygen. Aging at 50° C. in pure oxygen represents a fairly severe test regime, and materials which provide low SPME values (or little change in the SPME value compared to the initial SPME value) when so aged may provide very good protection under less stringent (e.g., room temperature) storage conditions. An SPME measurement is shown for example in the above-mentioned PCT Application Nos. PCT/US2009/030052 and PCT/US09/30054. The SPME value after 48 hours at 50° C. in pure oxygen may for example be less than 8,000, less than 5,000 or less than 4,000. The ratio of SPME after 48 hours at 50° C. to initial SPME may also be evaluated, and may for example be less than 8, less than 4, less than 2, less than 1.7 or less than 1.3.

The disclosed encapsulated materials may be used in a variety of products and applications including foods for human or animal consumption, e.g., beverages (for example dairy products including milk and yoghurt, and juice drinks including dry juice mixes), mixes (for example, baking mixes), prepared foods (for example, baked, frozen or precooked foods), food additives, food supplements, condiments (for example, barbecue sauce, mayonnaise, mustard or salad dressing), dietary supplements (for example, for use in weight maintenance or in nursing home care), nutritional snacks, nutritional supplements, neutraceuticals, medicines (for example, for maintaining heart health in humans and animals), and for non-food uses including catalysts, inks and coatings.

The invention is further described in the following Examples, in which all parts and percentages are by weight unless otherwise indicated.

### EXAMPLES

### Example 1

### Solubility Evaluations

The solubility of ARBORIS™ AS-2 phytosterol (from Arboris, LLC) in soybean cooking oil was evaluated by heating the oil to 150° C., fully dissolving a graded series of phytosterol concentrations (from 1 to 5 wt. %, in steps of 0.5 wt. %) in the heated oil, cooling the samples to room temperature and waiting 24 hours for any supersaturating phytosterol to crystallize. The phytosterol appeared soluble in room temperature cooking oil up to at least a concentration of 1.5 wt. %, and exhibited precipitates at concentrations at or above 2.0 wt. %. The results are set out below in Table 4:

TABLE 4

| Run | AS-2 | AS-2 | Oil | |
|---|---|---|---|---|
| No. | (wt. %) | (g) | (g) | Observations After Cooling |
| Ctrl. | 0.0 | 0.000 | 5.00 | Control-no apparent change |
| 1 | 0.5 | 0.025 | 4.98 | No apparent change |
| 2 | 1.0 | 0.050 | 4.95 | No apparent change |
| 3 | 2.0 | 0.100 | 4.90 | No apparent change |
| 4 | 3.0 | 0.150 | 4.85 | Long needle-like crystals, slight increase in viscosity |
| 5 | 4.0 | 0.200 | 4.80 | Cloudy-flocculent, increased viscosity |
| 6 | 5.0 | 0.250 | 4.75 | Same as above with slight increase in viscosity |
| 7 | 10.0 | 0.500 | 4.50 | Two phases: crust at surface, viscous oil below |
| 8 | 20.0 | 1.000 | 4.00 | Same as above with slight increase in oil viscosity |
| 9 | 40.0 | 2.000 | 3.00 | Solid wax-like but easily defotmed |
| 10 | 60.0 | 3.000 | 2.00 | Solid, harder than above |
| 11 | 80.0 | 4.000 | 1.00 | Solid, very hard |
| 12 | 90.0 | 4.500 | 0.50 | Solid, very hard |

Similar results were obtained when the phytosterol was added to ETERNA™ omega-3 fish oil (from Hormel) containing 2000 ppm tocopherol. The phytosterol appeared soluble in room temperature fish oil up to at least a concentration of 1.5 wt. %, and exhibited precipitates at concentrations at or above 2.0 wt. %. Above 10 wt. % phytosterols, the mixture was relatively solid in appearance.

In a further set of runs, CARDIOAID™ phytosterols (from Archer Daniels Midland) were added at eight different concentrations to heated omega-3 oil. For each run, a 5.0 g sample of the omega-3 oil was placed in a small aluminum weighing dish and heated at the lowest setting on a hot plate. The phytosterols were added to the hot oil and allowed to dissolve fully until there was no evidence of clouding or other insolubility. The samples were removed from the hot plate, allowed to cool to room temperature and observed about 18 hours later. The results are shown below in Table 5:

TABLE 5

| Run | CARDIOAID | CARDIOAID | Observations ~18 hours |
|---|---|---|---|
| No. | (g) | (wt. %) | After Cooling |
| Ctrl. | 0.00 | 0.00 | No apparent change in oil |
| 1 | 0.20 | 3.85 | Cloudy |
| 2 | 0.50 | 9.09 | Very soft gel, easily drips |
| 3 | 0.75 | 13.04 | Gel |
| 4 | 1.00 | 16.67 | Flowable paste |
| 5 | 1.25 | 20.00 | Soft paste, slight flow |
| 6 | 1.50 | 23.08 | Paste, moderately stiff |
| 7 | 0.05 | 0.99 | Solid granules apparent, |
| | | | little effect on viscosity |
| 8 | 0.10 | 1.96 | Grainy, increase in viscosity |

The phytosterol appeared soluble in room temperature fish oil up to at least a concentration of 1.5 wt. %, and exhibited precipitates at concentrations at or above 2.0 wt. %. Above 9 wt. % phytosterols, the mixture was relatively solid in appearance, and above 23 wt. % phytosterols a paste was formed.

### Example 2

### Prill Formation

Three different core formulations were prepared using the ingredients shown below in Table 6:

TABLE 6

| | | ARBORIS |
|---|---|---|
| | ETERNA | AS-2 |
| Formulation | Omega-3 Oil | Phytosterol |
| No. | (wt. %) | (wt. %) |
| 1 | 77 | 23 |
| 2 | 45.5 | 54.5 |
| 3 | 29.5 | 70.5 |

Stainless steel vessels on hotplates were used to melt 1 kg of each formulation. The oil was added to each vessel and the vessels were flushed with nitrogen and covered, then heated to about 150° C. The phytosterol was added slowly with stirring by hand, and each addition was allowed to melt fully before proceeding. The vessels were again flushed with nitrogen and covered before storing in a 150° C. oven. Prilling was completed within three hours of melting, by atomizing the formulations in a NIRO™ MOBIL MINOR™ spray drier (from GEA Niro) modified to supply liquid nitrogen to the drying chamber at a rate sufficient to maintain the exhaust temperature below about −20° C. The molten core formulations were passed through a spray nozzle into the focal point of the cool nitrogen causing rapid congealing and solidification of the spray into prilled core particles. After collection, each sample was immediately placed in a −20° C. freezer. The respective prill yields for Formulations 1 through 3 were 449 g (44.9%), 728 g (72.8%) and 778 g (77.8%). The samples were analyzed using Differential Scanning Calorimetry (DSC) to determine their melting point. The Formulation 1 through 3 prills had respective melting point values of 119.91, 122.76 and 127.11° C. The Formulation 2 and 3 prills were analyzed using a HORIBA™ LA-930 particle size analyzer (from Horiba Instruments, Inc.) to determine Particle Size Distribution (PSD) data, and found to have respective average particle diameters of 77.18 μm and 68 μm. The Formulation 1 prill formed an agglomerated mass that was not subjected to PSD analysis.

### Example 3

### Gelatin-Coated Omega-3/Phytosterol Prill

Using 1 kg of the Formulation 3 prilled cores, a gelatin protective layer could be applied as follows. A 20 g portion of 75 bloom gelatin may be added to 600 g of 80° C. deionized (DI) water and allowed to hydrate fully. A water bath may be used to cool the gelatin solution to 40° C. Using a WURSTER suspension coater equipped with a gaseous nitrogen drying feed, the prilled cores may be added to the solids fluidizer using a 340 l/min flow rate and the gelatin solution may be added to the liquids fluidizer using a 0.24 Mpa atomization pressure and 65° C. coating temperature. Free-flowing microparticles may be formed by removal of water from the gelatin-coated omega-3/phytosterol microparticles. After collection, the microparticles may be placed in a −20° C. freezer for storage. The microparticles should contain about 69.1 wt. % phytosterol, 28.9 wt. % omega-3 oil and 2 wt. % gelatin.

### Example 4

### Carnauba Wax-Coated Omega-3/Phytosterol Prill

Using 1 kg of the Formulation 2 prilled cores, a Carnauba wax protective layer could be applied as follows. A 200 g portion of Carnauba wax (melting point 82-86° C.) may be melted in a stainless steel vessel on a hotplate. The vessel may be flushed with nitrogen and covered prior to an initial heating to about 90° C. The Formulation 2 prilled cores may be stirred into the melted Carnauba wax. Re-prilling may be carried out using a modified NIRO MOBIL MINOR™ spray drier like that employed in Example 2 to form Carnauba wax-coated omega-3/phytosterol microparticles. After collection, the microparticles may be placed in a −20° C. freezer for storage. The microparticles should contain about 45.4 wt. % phytosterol, 37.9 wt. % omega-3 oil and 16.7 wt. % Carnauba wax.

### Example 5

### Stearic Acid-Coated Omega-3/Phytosterol Prill

Using 1 kg of the Formulation 2 prilled cores, a stearic acid protective layer could be applied as follows. A 50 g portion of stearic acid (melting point 70° C.) could be dry-blended with the Formulation 2 prilled cores for 5 minutes under high shear conditions in a WARING™ blender (from Waring Products, Inc.). The dry blending process should impart sufficient heat to the stearic acid and prilled cores to form stearic acid-coated omega-3/phytosterol microparticles. After collection, the microparticles may be placed in a −20° C. freezer for storage. The microparticles should contain about 51.9 wt. % phytosterol, 43.3 wt. % omega-3 oil and 4.8 wt. % stearic acid.

### Example 6

### Stearic Acid-Coated Omega-3/Phytosterol Prill

Using 1 kg of the Formulation 1 prilled cores and a procedure similar to that shown in Example 5, a stearic acid protective layer could be applied as follows. A 100 g portion of stearic acid (melting point 70° C.) could be dry-blended with the Formulation 1 prilled cores in a WARING™ blender for 5 minutes as in Example 5. The resulting stearic acid-coated omega-3/phytosterol microparticles may then be passed quickly through a hot air zone of at least 90° C. to ensure that the stearic acid surface forms an even coating on the omega-3/phytosterol prilled cores. After collection, the microparticles may be placed in a −20° C. freezer for storage. The microparticles should contain about 20.9 wt. % phytosterol, 70 wt. % omega-3 oil and 9.1 wt. % stearic acid.

The encapsulated materials in Examples 3 through 6 would contain the ingredient amounts shown below in Table 7:

TABLE 7

| | Oxidizable Oil, | Phytosterol, | Protective Shell, |
|---|---|---|---|
| Example | Wt. % | Wt. % | Wt. % |
| 3 | 28.9% | 69.1% | 2.0% gelatin |
| 4 | 37.9% | 45.4% | 16.7% Carnauba wax |
| 5 | 43.3% | 51.9% | 4.8% stearic acid |
| 6 | 70.0% | 20.9% | 9.1% stearic acid |

The disclosed invention involves a number of embodiments, including:

1. A method for protecting an oxidatively unstable material, which method comprises congealing droplets of a molten blend of oxidatively unstable material and phytosterol in a chilling gas stream to prilled cores comprising oxidatively unstable material and phytosterol, and encapsulating the prilled cores in one or more protective shell layers to form free-flowing microparticles.

2. The method of embodiment 1 wherein the oxidatively unstable material comprises a polyunsaturated fatty acid.

3. The method of embodiment 2 wherein the polyunsaturated fatty acid comprises an omega-3 or omega-6 fatty acid.

4. The method of embodiment 1 wherein the oxidatively unstable material comprises a triacylglycerol.

5. The method of embodiment 1 wherein the oxidatively unstable material comprises an antioxidant.

6. The method of embodiment 1 wherein the oxidatively unstable material comprises an acidulant, animal product, carotenoid, catalyst, drug, dye, enzyme, flavor, fragrance, lutein, lycopene, metal complex, natural color, nutraceutical, pigment, polyphenolic, processed plant material, metabiotic, probiotic, protein, squalene, tocopherol, tocotrienol, vitamin, unsaturated organic compound, or mixture thereof.

7. The method of embodiment 1 wherein the oxidatively unstable material is a solid at room temperature.

8. The method of embodiment 1 wherein the oxidatively unstable material is a gel at room temperature.

9. The method of embodiment 1 wherein the oxidatively unstable material is a liquid at room temperature.

10. The method of embodiment 1 comprising drying a protective shell layer.

11. The method of embodiment 1 comprising prilling a protective shell layer.

12. The method of embodiment 1 comprising forming a protective shell layer from gelatin.

13. The method of embodiment 1 comprising forming a protective shell layer from a triglyceride wax.

14. The method of embodiment 1 comprising forming a protective shell layer from stearic acid.

15. The method of embodiment 1 comprising forming a protective shell layer comprising insoluble or soluble fiber.

16. The method of embodiment 1 comprising forming a protective shell layer from a water-dispersible oxygen barrier layer, a hydrocolloid layer or a lipophilic layer.

17. The method of embodiment 1 wherein the encapsulated oxidatively unstable material has enhanced protection from oxidation compared to the oxidatively unstable material by itself.

18. The method of embodiment 1 further comprising combining the microparticles with a food to provide a food product.

19. The method of embodiment 18 wherein the food product when aged for three months has a minimal off taste profile.

20. The method of embodiment 18 wherein the food product is minimally processed.

21. An encapsulated material comprising free-flowing microparticles containing a prilled core comprising a congealed blend of oxidatively unstable material and phytosterol, covered by at least one protective shell layer.

22. The encapsulated material of embodiment 21 wherein the core comprises a polyunsaturated fatty acid.

23. The encapsulated material of embodiment 22 wherein the polyunsaturated fatty acid comprises an omega-3 or omega-6 fatty acid.

24. The encapsulated material of embodiment 23 wherein the core contains omega-3 fatty acid and phytosterol in a weight ratio of about 325:16 to about 8:3.

25. The encapsulated material of embodiment 21 wherein the core comprises a triacylglycerol.

26. The encapsulated material of embodiment 21 wherein the core comprises an antioxidant.

27. The encapsulated material of embodiment 26 wherein the antioxidant comprises Vitamin A, D, E, K or mixture thereof.

28. The encapsulated material of embodiment 26 wherein the antioxidant comprises ubiquinol, ubiquione or mixture thereof.

29. The encapsulated material of embodiment 21 wherein the core comprises an acidulant, animal product, carotenoid, catalyst, drug, dye, enzyme, flavor, fragrance, lutein, lycopene, metal complex, natural color, nutraceutical, pigment, polyphenolic, processed plant material, metabiotic, probiotic, protein, squalene, tocopherol, tocotrienol, vitamin, unsaturated organic compound, or mixture thereof.

30. The encapsulated material of embodiment 21 wherein the core is greater than 30 wt. % of the encapsulated material.

31. The encapsulated material of embodiment 21 wherein the oxidatively unstable material is a solid at room temperature.

32. The encapsulated material of embodiment 21 wherein the oxidatively unstable material is a gel at room temperature.

33. The encapsulated material of embodiment 21 wherein the oxidatively unstable material is a liquid at room temperature.

34. The encapsulated material of embodiment 21 wherein a protective shell layer comprises gelatin.

35. The encapsulated material of embodiment 21 wherein a protective shell layer comprises triglyceride wax.

36. The encapsulated material of embodiment 21 wherein a protective shell layer comprises stearic acid.

37. The encapsulated material of embodiment 11 wherein a protective shell layer comprises insoluble or soluble fiber.

38. The encapsulated material of embodiment 21 wherein a protective shell layer comprises a water-dispersible oxygen barrier layer, a hydrocolloid layer or a lipophilic layer.

39. The encapsulated material of embodiment 21 wherein the oxidatively unstable material has enhanced protection from oxidation compared to the oxidatively unstable material by itself.

40. A food product comprising the encapsulated material of embodiment 21.

41. The food product of embodiment 40 wherein the food product when aged for three months has a minimal off taste profile.

42. The food product of embodiment 40 wherein the food product is minimally processed.

43. The food product of embodiment 40 wherein the food is a baked food product.

44. The food product of embodiment 40 wherein the food is a nutritional snack.

45. The food product of embodiment 40 wherein the food is a dietary supplement.

Although specific examples, compositions, ingredients, temperatures and proportions have been disclosed in various aspects of the present invention, those disclosures are intended to be exemplary of species within a generic invention.

We claim:

1. A method for protecting an oxidatively unstable material, which method comprises congealing droplets of a molten blend of oxidatively unstable material and phytosterol in a chilling gas stream to form prilled cores comprising oxidatively unstable material and phytosterol, and encapsulating the prilled cores in one or more protective shell layers to form free-flowing microparticles.

2. The method of claim 1 wherein the oxidatively unstable material comprises a polyunsaturated fatty acid.

3. The method of claim 2 wherein the polyunsaturated fatty acid comprises an omega-3 or omega-6 fatty acid.

4. The method of claim 1 wherein the oxidatively unstable material comprises an acidulant, animal product, antioxidant, carotenoid, catalyst, drug, dye, enzyme, flavor, fragrance, lutein, lycopene, metal complex, natural color, nutraceutical, pigment, polyphenolic, processed plant material, metabiotic, probiotic, protein, squalene, tocopherol, tocotrienol, triacylglycerol, vitamin, unsaturated organic compound, or mixture thereof.

5. The method of claim 1 wherein the phytosterol comprises a non-esterified phytosterol.

6. The method of claim 1 comprising drying or prilling a protective shell layer.

7. The method of claim 1 comprising forming a protective shell layer from gelatin.

8. The method of claim 1 comprising forming a protective shell layer from triglyceride wax, stearic acid, insoluble fiber, soluble fiber, a water-dispersible oxygen barrier layer, a hydrocolloid layer or a lipophilic layer.

9. The method of claim 1 wherein the encapsulated oxidatively unstable material has enhanced protection from oxidation compared to the oxidatively unstable material by itself.

10. The method of claim 1 further comprising combining the microparticles with a food to provide a food product.

11. An encapsulated material comprising free-flowing microparticles containing a prilled core comprising a congealed blend of oxidatively unstable material and phytosterol, covered by at least one protective shell layer.

12. The encapsulated material of claim 11 wherein the core comprises a polyunsaturated fatty acid.

13. The encapsulated material of claim 12 wherein the polyunsaturated fatty acid comprises an omega-3 or omega-6 fatty acid.

14. The encapsulated material of claim 13 wherein the antioxidant comprises ubiquinol, ubiquione or mixture thereof.

15. The encapsulated material of claim 11 wherein the core comprises an acidulant, animal product, antioxidant, carotenoid, catalyst, drug, dye, enzyme, flavor, fragrance, lutein, lycopene, metal complex, natural color, nutraceutical, pigment, polyphenolic, processed plant material, metabiotic, probiotic, protein, squalene, tocopherol, tocotrienol, triacylglycerol, vitamin, unsaturated organic compound, or mixture thereof.

16. The encapsulated material of claim 11 wherein the phytosterol comprises a non-esterified phytosterol.

17. The encapsulated material of claim 11 wherein the core is greater than 30 wt. % of the encapsulated material.

18. The encapsulated material of claim 11 wherein the oxidatively unstable material is a solid, gel or liquid at room temperature.

19. The encapsulated material of claim 11 wherein a protective shell layer comprises gelatin.

20. The encapsulated material of claim 11 wherein a protective shell layer comprises triglyceride wax, stearic acid, insoluble fiber, soluble fiber, a water-dispersible oxygen barrier layer, a hydrocolloid layer or a lipophilic layer.

21. The encapsulated material of claim 11 wherein the oxidatively unstable material has enhanced protection from oxidation compared to the oxidatively unstable material by itself.

22. A food product comprising the encapsulated material of claim 11.
